(19) United States
(12) Patent Application Publication
(10) Pub. No.: US 2010/0111969 A1
(43) Pub. Date: May 6, 2010

(54) IL-4 RECEPTOR AND IL-13 AS PROGNOSTIC MARKERS FOR COLON AND PANCREAS TUMORS

(75) Inventors: Harald FRICKE, Heidelberg (DE); Marko KORNMANN, Neu Ulm (DE)

Correspondence Address: ROTHWELL, FIGG, ERNST & MANBECK, P.C., 1425 K STREET, N.W., SUITE 800, WASHINGTON, DC, 20005, US

(73) Assignee: Apogenix GmbH, Heidelberg (DE)

(21) Appl. No.: 12/609,309

(22) Filed: Oct. 30, 2009

Related U.S. Application Data
(60) Provisional application No. 61/110,044, filed on Oct. 31, 2008.

(51) Int. Cl. C07K 16/24; G01N 33/53; A61K 39/395

(52) U.S. Cl. 424/158.1; 435/7.92; 530/387.1

(57) ABSTRACT

The present invention relates to the use of IL-4 receptor and IL-13 expression as diagnostic and/or prognostic markers for tumors, such as colon and pancreas tumors.

### FIELD OF THE INVENTION

The present invention relates to the use of IL-4 receptor and IL-13 expression as diagnostic and/or prognostic markers for tumors, such as colon and pancreas tumors.

### STATE OF THE ART

Interleukin (IL)-13 is a secreted immunomodulatory cytokine with anti-inflammatory functions [1-3]. It regulates the expression of cytokines and adhesion molecules in several cell types, including colon-cancer cells [4-7]. Signaling is initiated by binding to the IL-13 receptor al chain (IL-13Rα1), which by itself has a low affinity to IL-13 and has no intracellular signaling capacity [8] but which forms a high-affinity receptor complex in association with the transmembrane IL-4 receptor-α (IL-4R) chain [8, 9]. A second IL-13 receptor chain termed IL-13Rα2 has also been identified [10]. In contrast to IL-13Rα1, IL-13Rα2 has a high affinity for IL-13, but appears to be a non-signaling decoy receptor. Although cytokine receptors do not exhibit catalytic tyrosine kinase activity in their cytoplasmic domains, they associate with cytoplasmic tyrosine kinases following ligand stimulation. Thus, IL-13 can lead to rapid activation of Janus and phosphatidylinositol (PI) 3-kinases and to the rapid phosphorylation of insulin receptor substrate (IRS) proteins [11, 12].

Pancreatic ductal adenocarcinoma (PDAC) is a lethal malignancy with a poor prognosis, in part due to its propensity to rapidly metastasize to lymph nodes and adjacent tissues while exhibiting resistance immune mechanisms directed against cancer [13]. PDACs frequently overexpress multiple growth factors and their receptors [14], as well as downstream signaling proteins, such as IRS-1 and IRS-2, which may also play an important role in mediating mitogenic signaling [15, 16]. PDACs also overexpress a number of cytokines and chemokines, such as interleukin-8 [17, 18]. Moreover, IL-4 is expressed and secreted by cultured human pancreatic cancer cells and exerts mitogenic effects via autocrine and paracrine mechanisms [19]. These cells also express IL-13Rα1 and IL-4R [20]. Although IL-13 was originally discovered in activated Th-2 lymphocytes and shares several biologic activities with IL-4 [1, 2] its potential role in PDAC has not been investigated despite the well-known proclivity of pancreatic cancer to spread to lymph nodes early and to avoid immune mechanisms directed against cancer.

Thus, there was a long-felt need for diagnostic and/or prognostic markers for tumors such as colon and pancreas tumors indicative of lymph node involvement and which would allow to estimate the overall survival of a tumor patient.

### SUMMARY OF THE INVENTION

It was discovered that IL-13 exhibits a growth-promoting role, in particular an autocrine growth-promoting role in pancreatic cancer cells and in colon cancer cells. Furthermore, IL-13 plays a role in immune modulation. It has further been found that a high expression of IL-4 receptor and/or IL-13 was associated with a high incidence of lymph node involvement and/or a reduced overall survival.

Thus, it was an object of the present invention to make use of IL-4 receptor and IL-13 expression as diagnostic and/or prognostic markers for tumors, preferably for solid tumors, and more preferably for colon and pancreas tumors.

A first preferred embodiment relates to a method for the diagnosis and/or prognosis of tumors, comprising the step of determining IL-4 receptor and/or IL-13 expression as diagnostic and/or prognostic marker in a patient.

In a preferred embodiment of the invention, high expression of IL-4 receptor and IL-13 is associated with a high incidence of lymph node involvement and/or a reduced overall survival.

In another preferred embodiment the pharmaceutical composition comprises as an active agent an IL-13 antagonist in combination with an IL-4 receptor antagonist for the treatment of tumors, preferably solid tumors, more preferably pancreas and/or colon tumors.

A further preferred embodiment of the invention relates to a method for the treatment of tumors, preferably solid tumors, more preferably pancreas and/or colon tumors, comprising administering a therapeutically effective amount of an IL-13 antagonist and/or an IL-4 receptor antagonist to a patient.

### DETAILED DESCRIPTION OF THE INVENTION

The effects of IL-13 on the growth and mitogenic signaling in cultured pancreatic cancer cells have been determined and IL-13 and IL-13 receptor expression in human PDAC have been characterized.

We determined that IL-13 is expressed and secreted by pancreatic cancer cells and enhances cell proliferation in association with activation of p44/42 mitogen-activated protein kinase (MAPK). We also demonstrated that high expression of IL-13 and IL-4R in R₀-resected primary PDAC are associated with an increased incidence of lymph node metastases. In addition we studied the expression of IL-4, IL-4R, IL-13, IL-13R in resected colon cancer. We demonstrate that high co-expression of IL-13 and IL-4R in the tumor is associated with reduced overall survival compared to patients not expressing the cytokine and the receptor.

IL-13 is an immunomodulatory and anti-inflammatory cytokine that exerts direct influence on B cells and the monocyte/macrophage population and is usually expressed in activated Th-2 lymphocytes [1, 2]. Although IL-13 is principally involved in the regulation of interactions between hematopoietic cell types [3], it also modulates a variety of functions in endothelial, mesothelial, and epithelial cell types [4-7, 23]. In addition, cell lines derived from Hodgkin's lymphoma and Reed-Sternberg cells in Hodgkin's lymphomas express IL-13 and IL-13 receptors and are growth stimulated by exogenous IL-13 [24, 25]. IL-13 receptors are also widely expressed in non-hematopoietic tissues, including heart, liver, skeletal muscle, ovary, and pancreas [8, 19]. We previously reported that human pancreatic cancer cell lines express various levels of the IL-13Rα1, IL-13Rα2, and IL-4R chains [20].

We demonstrated for the first time that IL-13 enhances pancreatic cancer cell proliferation and that these cells express and secrete IL-13. There was no correlation between the level of expression of the IL-13 receptor chains and IL-13 induced cell proliferation. IL-13 also enhanced the proliferation of a prostate cancer cell line [26] and inhibited apoptosis of HT-29 colon cancer cells in a PI 3-kinase dependent manner [27, 28]. In contrast, IL-13 inhibited both basal and estrogen-induced growth of cultured human breast cancer cell lines [29, 30] and anchorage-dependent and -independent growth of renal carcinoma cells [31]. Renal cell carcinoma cells are the only non-hematopoietic cell types which have also been shown to express IL-13 mRNA. In contrast to pancreatic cancer cells, however, these cells are growth inhibited by IL-13 [31]. Taken together, these observations indicate that IL-13 may exert divergent effects on proliferation in different cancer cell types.

Several lines of evidence suggest that IL-13 has the potential to exert autocrine growth stimulatory effects in some pancreatic cancer cell lines: 1) IL-13 stimulated pancreatic cancer cell growth, 2) pancreatic cancer cells expressed IL-13 at the mRNA and protein level and released IL-13 into conditioned medium, and 3) anti-IL-13 neutralizing antibodies partially inhibited the growth of two out of three cell lines that were growth stimulated by exogenous IL-13. Together with our observation that IL-13 was detectable in a great portion of primary PDACs and the previous report that pancreatic cancer cells express IL-4 receptor and IL-13 chains [20], these results raise the possibility that IL-13 may act in an autocrine manner to enhance the growth of pancreatic cancer cells in vivo.

The p44/42 MAPK pathway is a key component in the signal transduction of many mitogenic growth factors, such as IGF-I and EGF. Activation of the MAPK cascade was described for several cytokines, including IL-3, IL-4, IL-12, and IL-13 [5, 32]. We demonstrated that IL-13 activated p44/42 MAPK in pancreatic cancer cells. Two lines of evidence suggest that this activation was associated with growth stimulation, rather than inhibition, of apoptosis as reported for colon cancer cell lines [27, 28]. First, IL-13 resulted in a shift of cell cycle distribution enhancing the fraction of cells in the S and G₂ phases. Second, IL-13 failed to activate MAPK in the 3 cell lines that were resistant to IL-13 mediated growth stimulation.

PI 3-kinase has also been implicated in the signal transduction of many growth factors and cytokines that regulate cell proliferation and survival [32-34]. IL-13 activated PI 3-kinase in HT-29 colon [27, 28] and, in pancreatic cancer cells, in which it also induced tyrosine phosphorylation of IRS proteins. The IRS proteins may facilitate the coupling of the IL-13 receptor complex to the signaling cascades regulated by PI 3-kinase, as demonstrated for the IL-4Rα chain [12, 35, 36]. In the present study, IRS-2 associated PI 3-kinase activity was enhanced by IL-13 in all 6 pancreatic cancer cell lines. In view of the observations that tyrosine phosphorylation of IRS-2 may lead to enhanced mitogenic signaling [12] and that IRS-2 is over-expressed in pancreatic cancer [15], it is possible that IRS-2 contributes to the mitogenic signaling pathway that is activated by IL-13 in pancreatic cancer cells by facilitating PI 3-kinase activation. However, inasmuch as IL-13 only activated p44/42 MAPK in the growth responsive cells, it is probable that activation of PI 3-kinase per se may not be sufficient to activate mitogenesis in pancreatic cancer cells. IL-13 can attenuate Th1-cell-driven, cell-mediated immune responses through its effects on the monocyte/macrophage cells, causing down-regulation of pro-inflammatory cytokines, like IL-1, IL-6, and TNF-α, and chemokines, like IL-8 and MIP-1α, and inhibiting antibody-dependent cellular cytotoxicity [3, 25]. IL-13 also acts on fibroblasts and mediates fibrosis in allergic asthma and Schistosoma mansoni-induced hepatic fibrosis [37, 38]. Immunohistochemical analysis of PDAC and colon cancer samples revealed the presence of moderate to strong cytoplasmic IL-13 immunoreactivity in the cancer cells within the tumor mass. Although there was no clear association between IL-13 protein expression and patient prognosis in this group, our analysis revealed that co-expression of IL-13 and IL-4R was associated with a higher frequency of lymph node metastases in PDAC and reduced overall survival in PDAC and colon cancer. This observation raises the possibility that IL-13, in addition to exerting direct effects on pancreatic cancer cell proliferation in the presence of IL-4R, may contribute to the inhibition of immune mechanisms directed against pancreatic cancer, thereby facilitating tumor spread. Several lines of evidence support this possibility. Thus, IL-4R and signal transducer and activator of transcription 6 (STAT6) knockout mice resisted tumor recurrence, whereas IL-4 knockout mice exhibited tumor recurrence [39]. However, injection of soluble IL-13 receptors into the IL-4 knockout mice suppressed tumor recurrence, whereas the loss of natural killer T cells (NKT cells) in CD1 knockout mice resulted in decreased IL-13 production and also induced resistance to tumor recurrence [39], indicating that IL-13 may be an essential player in this process. Furthermore, studies with STAT6 knockout mice indicated that metastasis is partially dependent on IL-13 and interferon-gamma [40]. A cytotoxin coupled to IL-13 moreover showed potent antitumor activity in human pancreatic tumors engineered to overexpress IL-13 receptor chains [41]. Together, these observations underscore the importance of IL-13 as an important suppressor of cancer-directed immunosurveillance and raise the possibility that pancreatic-cancer-cell derived IL-13 may also act in a paracrine manner to down-regulate tumor immunosurveillance, thereby facilitating metastasis. In view of the potential autocrine growth-promoting role of IL-13 in pancreatic cancer cells and its role in immunomodulation, it is possible that modalities which use this pathway [41] or interrupt this autocrine/paracrine loop [42] may have a therapeutic potential in PDAC in the future.

A first preferred embodiment relates to a method for the diagnosis and/or prognosis of tumors, comprising the step of determining IL-4 receptor and/or IL-13 expression as diagnostic and/or prognostic marker in a patient. Said patient may be suspected to suffer from a tumor, preferably a pancreas and/or colon tumor. Alternatively, the tumor, preferably a pancreas or colon tumor, may have already been diagnosed in said patient.

In one embodiment, IL-4 receptor and/or IL-13 expression is determined directly in a patient.

In another embodiment, IL-4 receptor and/or IL-13 expression is determined in a sample from a patient, wherein the sample is a biological sample in the form of body fluids, such as blood, serum, plasma, urine, cerebrospinal fluid, sweat, or in the form of a tissue sample, e.g. tissue exudate, tissue homogenate, or in the form of a histological sample.

According to the invention, high expression of IL-13 is associated with a high incidence of lymph node involvement and low expression of IL-13 is associated with a low incidence of lymph node involvement.

Further, according to the invention, high expression of IL-4 receptor is associated with a high incidence of lymph node involvement and low expression of IL-4 receptor is associated with a low incidence of lymph node involvement in PDAC.

In a preferred embodiment of the invention, high expression of IL-4 receptor and IL-13 is associated with a high incidence of lymph node involvement and/or a reduced survival rate, and low expression of IL-4 receptor and IL-13 is associated with a low incidence of lymph node involvement and an improved overall survival.

A further preferred embodiment of the invention relates to a pharmaceutical composition comprising as an active agent an IL-13 antagonist for the treatment of tumors, preferably solid tumors, more preferably pancreas and/or colon tumors.

Another preferred embodiment of the invention relates to a pharmaceutical composition comprising as an active agent an IL-4 receptor antagonist for the treatment of tumors, preferably solid tumors, more preferably pancreas and/or colon tumors.

In another preferred embodiment the pharmaceutical composition comprises as an active agent an IL-13 antagonist in combination with an IL-4 receptor antagonist for the treatment of tumors, preferably solid tumors, more preferably pancreas and/or colon tumors.

It is understood that said pharmaceutical composition may further comprise one or more pharmaceutically acceptable excipients. It is further understood that said pharmaceutical composition may comprise one or more additional active substances. In a preferred embodiment, the IL-4 receptor antagonist is selected from the group consisting of IL-4 receptor antibodies as described in EP 08 006 750.7, IL-4 muteins as described in WO 2008/003514 and/or other IL-4 receptor blocking agents.

A further preferred embodiment of the invention relates to a method for the treatment of tumors, preferably solid tumors, more preferably pancreas and/or colon tumors, comprising administering a therapeutically effective amount of an IL-13 antagonist and/or an IL-4 receptor antagonist to a patient. Those of ordinary skill in the art will be able to determine a suitable therapeutically effective amount based on the age, body weight, physical condition. The weekly dose of the inhibitor is preferably in the range of 0.1 mg/kg to 10 mg/kg, more preferably 1 mg/kg to 5 mg/kg, most preferably about 2 mg/kg. The administration is carried out for a time period sufficient to obtain the desired beneficial effect, e.g. induction of a tumor response to treatment.

The IL-13 antagonist and/or IL-4 receptor antagonist is preferably administered in combination with further anti-tumor therapy, e.g. radiation therapy and/or with at least one further medicament, e.g. a chemotherapeutic agent and/or an anti-tumor antibody.

If the treatment scheme comprises surgical removal of the tumor, the IL-13 antagonist and/or IL-4 receptor antagonist may be administered before, during and/or after surgery to the patient.

Another preferred embodiment of the invention relates to a method for the assessment of the longterm survival of an individual having a solid tumor such as pancreas and/or colon tumors. Said method comprises the steps of
(i) obtaining a sample of said solid tumor;
(ii) determining the level of expression of IL-13 and IL-4R in the tumor tissue; and
(iii) assessing the longterm survival of such individual based on the levels of expression of IL-13 and IL-4R in the tumor tissue,
   wherein simultaneous expression of IL-13 and IL-4 at high levels in the tumor tissue is indicative of reduced longterm survival of the individual.

The sample of the solid tumor preferably is a tissue sample, e.g. tissue exodate, tissue homogenate or a histological sample.

According to the invention, the simultaneous expression of IL-13 and IL-4 at high levels in the tumor tissue is associated with a high incidence of lymph node involvement and thus a reduced longterm survival rate.

It should be noted that the disclosure of all patent and non-patent documents recited in the specification above is incorporated herein by reference.

The present invention shall be illustrated further by the following examples.

### DESCRIPTION OF THE FIGURES

FIG. 1 Effect of IL-13 and IGF-I on cell growth and cell cycle. (a) IL-13 time course (cell counting). ASPC-1 (circle), CAPAN-1 (square), and COLO-357 (triangle) cells were seeded in 12-well plates and grown overnight in complete medium followed by serum-free medium for the indicated times in the presence or absence of IL-13 (5 nM). Medium was changed daily. Results are the means±SEM of triple determinations from 3 separate experiments. (*) p<0.05 compared to respective untreated controls. (b) IL-13 and IGF-I MTT assay. Indicated cells were incubated for 48 h in the absence or presence of 5 nM IL-13 (open bars) or IGF-I (solid bars). Results are the means±SEM of quadruple determinations from 3 separate experiments. (*) p<0.05 compared to respective untreated controls. (c) Cell cycle analysis. Cells were serum-starved for 24 h and then incubated in the presence or absence of IL-13 (5 nM, open bars) or IGF-I (5 nM, solid bars) for 24 h prior to analysis using a FACScan analysis system. Effects on cell cycle distribution are shown as relative difference of cell-cycle fraction in % compared to corresponding untreated controls and are means±SD of at least 5 separate experiments. (*) p<0.04 compared to untreated controls.

FIG. 2 Effects of IL-13 and IGF-I on p44/42 MAPK phosphorylation. Indicated cells were serum-starved for 18 h and then incubated for 5 min in the absence (−) or presence (+) of IL-13 (5 nM) or IGF-I (5 nM). Immunoblot analysis of total cell lysates was carried out with a phospho-p44/42 MAPK antibody (p-MAPK). To confirm specificity and equal loading, membranes were stripped and re-probed with a pan-ERK-2 antibody (ERK-2).

FIG. 3 The effect of IL-13 on IRS-2 associated PI 3-kinase activity. PI 3-kinase activity of IRS-2 immunoprecipitates from the indicated cells was determined after serum starvation and incubation (5 min) in the absence (−) or presence (+) of IL-13 (5 nM). The origin of the spotted samples and the migration of the phosphorylated phosphatidylinositol substrate (PI) are marked.

FIG. 4 IL-13 expression and secretion in pancreatic cancer cells. (a) mRNA expression. Northern blotting of poly(A)⁺ RNA (2 μg/lane) from the indicated cells was carried out using a human IL-13 riboprobe. β-actin was used as a loading control. rRNA markers are shown on the left. (b) Protein expression. Cell lysates were subjected to a IL-13 sandwich ELISA. Results are shown as IL-13 protein concentration per μg of cell lysate and are means±SD of 3 separate experiments. (c) IL-13 secretion. Cells were cultured for 48 h in serum-free medium supplemented with proteinase inhibitors. Medium was harvested and subjected to IL-13 ELISA. Results are shown as IL-13 protein concentration in 1 ml of conditioned medium.

FIG. 5 Effects of IL-13 neutralizing antibodies on pancreatic cancer cell growth. Results are shown as means±SEM of quadruple determinations from at least 3 separate experiments. (a) BaseI cell growth. ASPC-1 (solid circle), CAPAN-1 (solid square), and COLO-357 (solid triangle) cells (10,000/well) were grown in 96-well plates for 24 h in complete medium and then incubated for 48 h in the absence or presence of increasing concentrations of carrier-free neutralizing IL-13 antibody. (*) p<0.05 compared to control. (b) IL-13 stimulated growth. CAPAN-1 cells were incubated in serum-free medium for 48 h in the absence or presence of 10 μg/ml neutralizing IL-13 antibody (MAB), 5 nM IL-13 (IL-13), or the combination of both (MAB+IL-13) preincubated for 60 min at 37° C. (*) p<0.05 compared to IL-13 alone.

FIG. 6 IL-13 and IL-4R expression in human pancreatic cancer tissue samples. Moderate to strong IL-13 (a, c) and IL-4R (b, d) immunoreactivity was observed in ductal-like cancer cells in 30 of 70 and 28 of 70 samples, respectively. No clear IL-13, but IL-4R immunoreactivity was present in the nerve structures, as depicted by arrowheads (a, b). The arrows (a, b) indicate the areas of magnification for IL-13 (c) and IL-4R (d). IL-13 reactivity appeared in a fine granular pattern in the cytoplasm of the cancer cells (c). In most samples no or only faint IL-13 (e) and IL-4R (f) immunoreactivity was present. Original magnification: a, b, e, f 50×, c and d 200×.

### EXAMPLES

### Example 1

### Materials and methods

### Materials

The following were purchased: IL-13 ELISA kit and mouse monoclonal antibody against IL-13 from BioSource International (Camarillo, Calif.); Dako ABC kit for immunohistochemistry from Dako Denmark (Glostrup, Denmark); carrier-free neutralizing human IL-13 antibody (mouse monoclonal, 32116) and mouse monoclonal antibody against IL-4R from R&D Systems, Inc. (Minneapolis, Minn.); human IL-13, anti-active MAPK (p-ERK, E-4) mouse monoclonal antibody, and human recombinant IGF-I from Sigma Chemical Co. (St. Louis, Mo.); rabbit polyclonal antibodies against IRS-1 and IRS-2 from Upstate Biotechnology Inc. (Lake Placid, N.Y.).

### Cell Culture and Growth Assays

ASPC-1, CAPAN-1, MIA PaCa-2, and PANC-1 human pancreatic cancer cells were purchased from American Type Culture Collection (ATCC, Rockville, Md.). COLO-357 and T3M4 human pancreatic cancer cells were a gift from R. S. Metzgar (Duke University, Durham, N.C.). COLO-357, MIA PaCa-2, and PANC-1 cells were grown in DME medium, whereas ASPC-1, CAPAN-1, and T3M4 cells were grown in RPMI medium. All media were supplemented with 8% FBS, penicillin G (100 U/ml), and streptomycin (100 μg/ml), termed complete medium. Cells were maintained at 37° C. in humidified air with 5% CO₂.

The colorimetric 3-(4,5-Dimethylthiazol-2-yl)-2,5-diphenyltetrazolium bromide (MTT) proliferation assay was carried out as described previously [19]. Briefly, to assess the effects of IL-13 and IGF-I on cell growth, cells were cultured for 48 h in serum-free medium in the absence or presence of the indicated substrates before analysis. To assess the effects of neutralizing IL-13 antibodies on basal growth, cells were cultured in complete medium for 48 h. A CD5 antibody was used as negative control (Oncogene Science, Uniondale, N.Y.).

For cell counting, cells (75,000/well for ASPC-1 or CAPAN-1 and 50,000/well for COLO-357 cells) were seeded in 12-well plates. After 24 h, complete medium was replaced by serum-free medium for the indicated times in the absence or presence of IL-13. The medium was changed daily, including the respective additions.

### Cell Cycle Analysis

Flow cytometric analysis was performed by propidium iodine using a CycleTest Plus kit according to the manufacturer's instructions and a FACScan (Becton Dickinson) analysis system equipped with a FACStation, MAC PowerPC computer and CellQuest acquisition software as described [19].

### Mitogen-Activated Protein Kinase (MAPK) Phosphorylation

Cell extracts were prepared as described and subjected to immunoblot analysis using phospho-specific p44/42 MAPK or anti-pan ERK2 antibodies [21]. To estimate the magnitude of p44/42 MAPK phosphorylation, bands were scanned and normalized to pan-ERK2. Densitometric analysis was performed using Array Vision (Imaging Research Inc., Ontario, Canada).

### Immunoprecipitation and PI 3-Kinase Activity Assay

IRS immunoprecipitations and PI 3-kinase activity assays were carried out as described [15]. Briefly, cells were serum-starved for 18 h before the respective treatment. Lysates (1000 μg/1000 μl) were incubated with the respective primary antibody followed by precipitation with protein A sepharose and immunoblot analysis.

For the determination of PI 3-kinase activity [15], immunoprecipitates were sequentially washed followed by the in vitro PI 3-kinase reaction using PI substrate (0.5 mg/ml) and 10 nCi/μl [γ-32P]ATP for 5 min. Phospholipids were separated by thin-layer chromatography and exposed to films at −80° C. using intensifying screens.

Preparation of the IL-13 cDNA and Northern Blot Analysis

A 122 by human IL-13 cDNA fragment corresponding to nt 336 to 457 of the human IL-13 cDNA sequence (Genbank accession: X69079) [1] was generated by RT-PCR from human placenta RNA. The 122 fragment was subcloned into pBSK and its authenticy confirmed by sequencing. Northern Blot analysis of poly(A) RNA (2 μg) from exponentially growing cells and human placenta was carried out as described [20]. The IL-13 cRNA probe was labeled with [α-³²P]CTP using T7 RNA polymerase and the 13-actin cDNA probe was random-primed labeled with [α-³²P]dCTP.

### IL-13 ELISA

Total cell lysates (75 μg in 100 μl of lysis buffer) and 100 μl conditioned medium were used for the IL-13 ELISA. For the preparation of conditioned medium, subconfluent cells were incubated in 6-well plates for 48 h in serum-free medium containing the proteinase inhibitors aprotinin (50 μg/ml), benzamidine (10 μg/ml), leupeptin (10 μg/ml), and pepstatin A (10 μg/ml). After collection of the cell supernatant and centrifugation for 10 min at 1000 g, 100 μl aliquots were used for the IL-13 sandwich ELISA carried out according to the protocol of the manufacturer (BioSource International, Camarillo, Calif.). In our study, the inter-assay variability was less than 36% and the intra-assay variability less than 7%.

### Tissue Samples and Immunohistochemistry

Primary tumor specimens from 70 patients with R₀-resected PDAC were evaluable for IL-13 and IL-4R staining. Clinical and pathological information was obtained from the hospital tumor registry. The study was approved by the Ethics Committee of the University of Ulm (Nr. 105/98).

Formalin-fixed and paraffin-embedded 5 μm sections were stained using the streptavidin-peroxidase technique using a Dako ABC kit as described [22]. After deparaffinization and blocking endogenous peroxidase activity, the sections were incubated for 20 min at 23° C. with 1% normal horse serum and for 24 h at 4° C. with the specific monoclonal antibodies against human IL-13 (1:500) and IL-4R (1:4000). Bound antibodies were detected with biotinylated horse universal secondary antibodies and streptavidin-peroxidase complex, using diaminobenzidine tetrahydrochloride as the substrate. Sections were counterstained with Mayer's hematoxylin. Omission of primary antibodies did not yield any immunoreactivity.

All sections were examined independently by two observers (A.F., M.K.) blinded to both clinical and pathological data. Protein expression in the tumor cells was quantified using a visual grading system based on the intensity of staining from 0 to 3 [22]. The highest staining intensity found in tumor cells was used for classification. According to the staining intensity, cancers were classified as low (0+1) or high (2+3) expressing tumors. Staining intensity was judged in 73% and 76% of the cases similarly into 0, 1, 2, or 3 for IL-13 and IL-4R, respectively. The remaining cases were examined a second time together to obtain a final classification.

### Statistical Analysis

Results for cell growth and cell cycle are shown as mean±SEM or mean±SD. When indicated, Student's t-test or rank sum test were used for statistical analysis. Absolute and relative frequencies were calculated and compared using the Chi-Square or Fisher-Exact Test. Overall survival time was defined as the time from the day of operation to death (failure) or until data evaluation for the patients being alive or lost to follow up (censored). Kaplan-Meier plots were drawn and survival of the subgroups was analyzed using the log-rank test. Univariate analysis of survival was performed using SPSS software (Chicago, Ill.). P<0.05 was taken as level of significance (two-sided).

### Example 2

### Effect of IL-13 on Pancreatic Cancer-Cell Proliferation

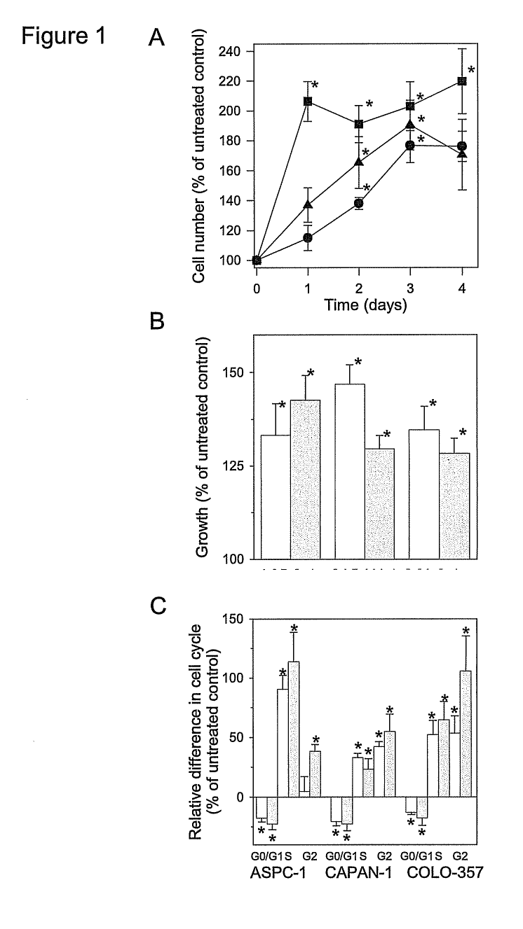

IL-13 exerted a dose-dependent increase in the growth in ASPC-1, CAPAN-1, and COLO-357 cells. At a maximally effective concentration of 5 nM, IL-13 increased cell growth by 33% (±2% SEM) and 35% (±2% SEM) in ASPC-1 and COLO-357 cells, respectively, after 48 h. In the most responsive CAPAN-1 cells, the stimulatory effect was further increased at 10 nM IL-13 to 51% (±4% SEM) after 48 h. To validate the results of the MTT assay and to characterize its effects during longer incubation periods, cell counting was performed in the absence and presence of 5 nM IL-13 for up to 4 days (FIG. 1).

IGF-I is a well-characterized mitogenic growth factor [14, 15]. The effects of IL-13 and IGF-I on proliferation and cell cycle were compared next. IGF-I and IL-13 enhanced proliferation in all three cell lines in the MTT assay after 48 h in association with increased S-phase cell fraction and reduced percentage of cells in Gₒ/G₁ (FIG. 1). CAPAN-1 cells showed best response to proliferation, but a relatively low fraction of cells in S-phase after 24 h. Most CAPAN-1 cells may have passed S-phase already after 24 h. IL-13 also increased the percentage of cells in G₂ in CAPAN-1 and COLO-357 cells.

In contrast to ASPC-1, CAPAN-1, and COLO-357 cells, IL-13 did not alter the growth of MIA PaCa-2, PANC-1, or T3M4 pancreatic cancer cells. IL-13-induced proliferation did not depend on IL-13 or IL-4 receptor expression. Relative expression levels of the six cell lines were determined earlier [20] and are summarized in Table 1.

TABLE 1
Expression of IL-4 receptor and IL-13 chains and the effect of IL-13
on cell proliferation in cultured human pancreatic cancer cell lines

| | Cell Lines | | | | | |
|---|---|---|---|---|---|---|
| | | | COLO- | MIA | PANC- | |
| | ASPC-1 | CAPAN-1 | 357 | PaCa-2 | 1 | T4M4 |
| Growth | ++ | +++ | ++ | − | − | − |
| enhancement | | | | | | |
| IL-13Rα1* | +++ | ++ | ++ | +++ | +++ | + |
| IL-13Rα2* | − | − | − | ++ | − | − |
| IL-4R* | +++ | ++ | +++ | + | ++ | +++ |

*Semi-quantitative mRNA expression levels of the IL-receptor chains were adopted from [20].

### Example 3

### Effect of IL-13 on p44/42 MAPKs

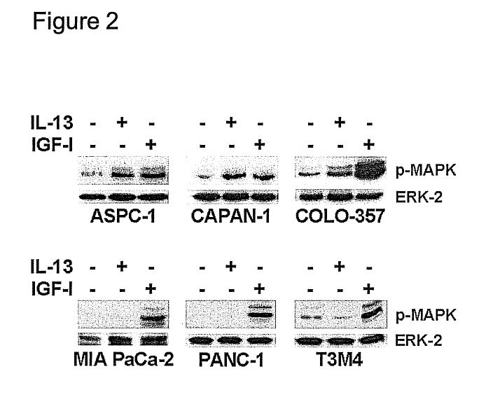

P44/42 MAPKs mediate mitogenic signaling of several growth factors [5, 14, 19]. IL-13 and IGF-I enhanced p44/42 MAPK phosphorylation in ASPC-1, CAPAN-1, and COLO-357 cells (FIG. 2). Densitometric analysis of multiple experiments (n=5) revealed an average increase of p44/42 MAPK phosphorylation by IL-13 of 98%, 82%, and 55% in ASPC-1, CAPAN-1, and COLO-357 cells, respectively. IL-13 did not enhance p44/42 MAPK phosphorylation in MIA PaCa-2, PANC-1, and T3M4 cells (FIG. 2). In contrast, IGF-I (FIG. 2) also enhanced p44/42 MAPK phosphorylation in IL-13-unreseponsive cells, indicating that the failure of IL-13 to activate MAPK in these cells was not due to a defective p44/42 MAPK cascade.

### Example 4

### Effect of IL-13 on Phosphatidylinositol (Pi) 3-Kinase Activity

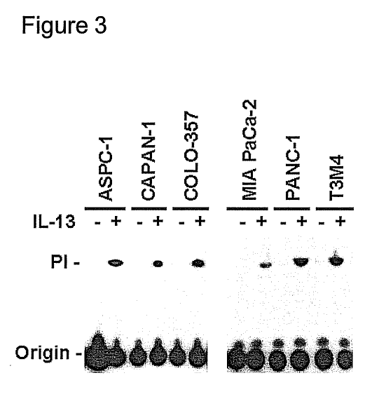

PI 3-kinase has also been implicated in cytokine-induced mitogenic signaling of several hematopoietic cell lines followed by strong tyrosine phosphorylation of IRS-1 and ±2. IL-13- and IGF-1-induced tyrosine phosphorylation of IRS-1 and -2 in all 6 cell lines (data not shown). Furthermore, IL-13 enhanced PI 3-kinase activity in the responsive, as well as in the unresponsive, cell lines (FIG. 3).

### Example 5

### Expression of IL-13 in Human Pancreatic Cancer Cell Lines

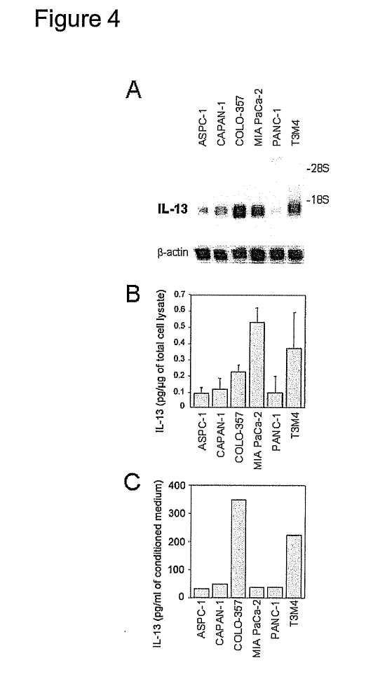

In view of the mitogenic effects of IL-13 in several of our cell lines, we investigated whether these cells express this cytokine. Northern blotting revealed the presence of the 1.4 kb IL-13 mRNA transcript [1] in all 6 cell lines (FIG. 4). IL-13 protein was detectable in total cell lysates and in conditioned medium by ELISA, indicating that these cells secrete IL-13 (FIG. 4).

### Example 6

### Effects of Neutralizing IL-13 Antibodies on Cell Growth

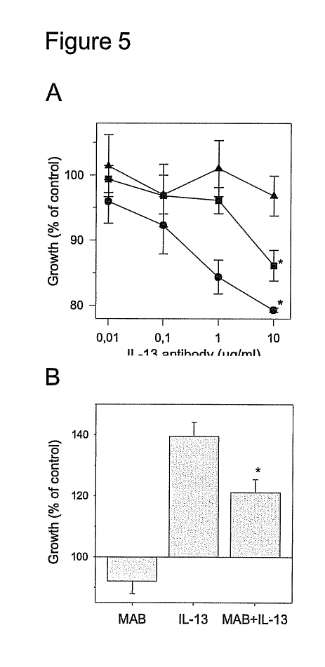

The effects of a neutralizing IL-13 antibody on basal cell growth were determined next. Incubating ASPC-1 and CAPAN-1 cells with increasing concentrations of the IL-13 neutralizing antibody inhibited cell growth in a dose-dependent manner (FIG. 5). The effects were small, but reproducible. By contrast, the basal growth of COLO-357 cells, which released highest levels of IL-13 into the medium, was not altered (FIG. 5A). Pre-incubation of IL-13 (5 nM) with the neutralizing IL-13 antibody (10 μg/ml) significantly reduced the mitogenic activity of IL-13 in CAPAN-1 cells (FIG. 5B). The neutralizing IL-13 antibody (10 μg/ml) failed to alter the growth of the three IL-13-unresponsive cell lines (data not shown). Additionally, a CD5 control antibody (10 μg/ml) did not alter the growth of any of the 6 cell lines (data not shown).

### Example 7

### IL-13 and IL-13 Receptor Expression in Pancreatic Cancer Tissues

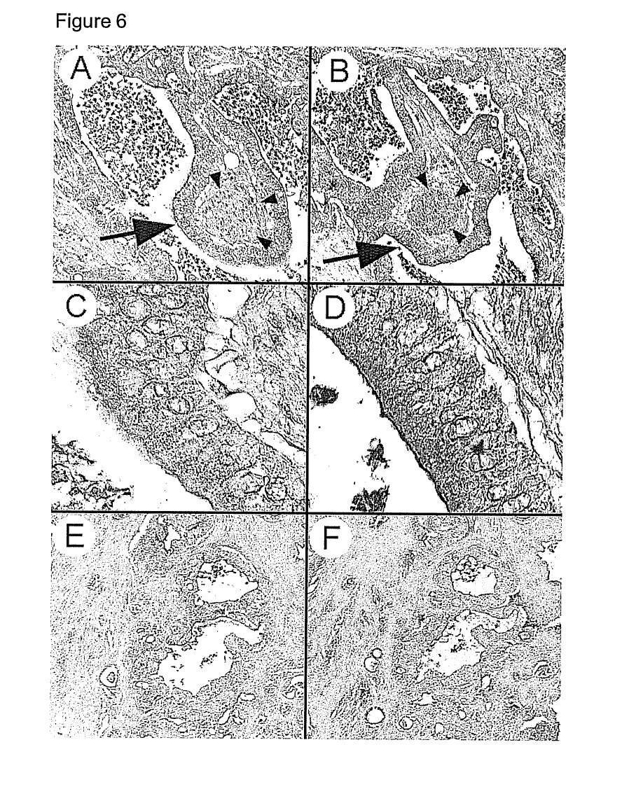

Immunohistochemical analysis was performed to determine the localization of IL-13 and its transmembrane receptor chain (IL-4R) in pancreatic cancer tissues. IL-13 immunoreactivity was not present in normal ductal, acinar, and islet cells (data not shown). Faint IL-4R immunoreactivity was present in some acinar, ductal, and islet cells of the normal pancreas (data not shown). High IL-13 and IL-4R immunoreactivity was present in the ductal cancer cells in 43% (30/70) and 40% (28/70) of the primary PDAC samples, respectively (FIG. 6).

Patient characteristics are summarized in Table 2. Analysis of IL-13 and IL-4R levels revealed that high IL-13 and IL-4R immunoreactivity was associated with positive lymph node involvement (Table 3). No significant differences in survival were observed among the subgroups with median survival times ranging from 16 to 19 months. However, long-term survival (more than 3 years) was not observed in any of the 16 patients whose tumors exhibited high IL-13 and IL-4R staining intensity. By contrast, the 3-year overall survival was 24% (13/54) in patients whose tumors exhibited low levels of immunoreactivity for either IL-13 or IL-4R (Table 3).

TABLE 2
Characteristics and clinical treatment of patients with pancreatic
cancer enrolled in IL-13 and IL-4R immunohistochemical analysis

| Characteristics | Patients (n = 70) |
|---|---|
| Median age (years) | 64 (34-77) |
| Age | |
| ≦60 | 23 |
| >60 | 47 |
| Gender | |
| male | 32 |
| female | 38 |
| Histology | |
| Ductal adenocarcinoma | 70 |
| Stage (UICC) | |
| I | 5 |
| II | 20 |
| III | 45 |
| Histologic grade | |
| 1 | 8 |
| 2 | 33 |
| 3 | 27 |
| 4 | 2 |
| Operative procedures | |
| pancreatoduodenectomy | 60 |
| distal pancreatectomy | 7 |
| total pancreatectomy | 3 |
| Adjuvant treatment | |
| none | 14 |
| intravenous chemotherapy | 19 |
| intra-arterial chemotherapy | 15 |
| intra-arterial chemotherapy + | 19 |
| radiotherapy | |
| not stated | 3 |

TABLE 3
Association of IL-13 and IL-4R expression with lymph node
involvement and the 3-year overall survival

| | | lymph node | | | Overall | | |
|---|---|---|---|---|---|---|---|
| Staining | | Involvement | | | Survival | | |
| intensity | N | negative | positive | P | ≦36 months | >36 months | P |
| IL-13 | | | | | | | |
| Low (0 + 1) | 40 | 19 | 21 | 0.0 | 32 | 8 | |
| High (2 + 3) | 30 | 6 | 24 | 36 | 25 | 5 | 0.967 |
| IL-4R | | | | | | | |
| Low (0 + 1) | 42 | 20 | 22 | 0.0 | 32 | 10 | |
| High (2 + 3) | 28 | 5 | 23 | 23 | 25 | 3 | 0.289 |
| IL-13 and IL-4R | | | | | | | |
| At least one low | 54 | 24 | 30 | 0.0 | 41 | 13 | |
| Both high | 16 | 1 | 15 | 13 | 16 | 0 | 0.075 |

### REFERENCES

1. Minty A, Chalon P, Derocq J M, Dumont X, Guillemot J C, Kaghad M, Labit C, Leplatois P, Liauzun P, Miloux B, Minty C, Casellas P, Loison G, Lupker J, Shire D, Ferrara P, Caput D (1993) Interleukin-13 is a new human lymphokine regulating inflammatory and immune responses. Nature 362:248-250
2. McKenzie A N, Culpepper J A, de Waal Malefyt R, Briere F, Punnonen J, Aversa G, Sato A, Dang W, Cocks B G, Menon S, De Vries J E, Banchereau J, Zurawski G (1993) Interleukin 13, a T-cell-derived cytokine that regulates human monocyte and B-cell function. Proc Natl Acad Sci USA 90:3735-3739
3. Zurawski G, de Vries J E (1994) Interleukin 13, an interleukin 4-like cytokine that acts on monocytes and B cells, but not on T cells. Immunol Today 15:19-26
4. Lind M, Deleuran B, Yssel H, Fink-Eriksen E, Thestrup-Pedersen K (1995) IL-4 and IL-13, but not IL-10, are chemotactic factors for human osteoblasts. Cytokine 7:78-82
5. David M, Ford D, Bertoglio J, Maizel A L, Pierre J (2001) Induction of the IL-13 receptor alpha2-chain by IL-4 and IL-13 in human keratinocytes: involvement of STAT6, ERK and p38 MAPK pathways. Oncogene 20:6660-6668
6. Sironi M, Sciacca F L, Matteucci C, Conni M, Vecchi A, Bernasconi S, Minty A, Caput D, Ferrara P, Colotta F (1994) Regulation of endothelial and mesothelial cell function by interleukin-13: selective induction of vascular cell adhesion molecule-1 and amplification of interleukin-6 production. Blood 84:1913-1921
7. Kanai T, Watanabe M, Hayashi A, Nakazawa A, Yajima T, Okazawa A, Yamazaki M, Ishii H, Hibi T (2000) Regulatory effect of interleukin-4 and interleukin-13 on colon cancer cell adhesion. Br J Cancer 82:1717-1723
8. Aman M J, Tayebi N, Obiri N I, Puri R K, Modi W S, Leonard W J (1996) cDNA cloning and characterization of the human interleukin 13 receptor alpha chain. J Biol Chem 271:29265-29270
9. Idzerda R L, March C J, Mosley B, Lyman S D, Vanden Bos T, Gimpel S D, Din W S, Grabstein K H, Widmer M B, Park L S (1990) Human interleukin 4 receptor confers biological responsiveness and defines a novel receptor superfamily. J Exp Med 171:861-873
10. Caput D, Laurent P, Kaghad M, Lelias J M, Lefort S, Vita N, Ferrara P (1996) Cloning and characterization of a specific interleukin (IL)-13 binding protein structurally related to the IL-5 receptor alpha chain. J Biol Chem 271:16921-16926
11. Jiang H, Harris M B, Rothman P (2000) IL-4/IL-13 signaling beyond JAK/STAT. J Allergy Clin Immunol 105:1063-1070
12. White M F, Yenush L (1998) The IRS-signaling system: a network of docking proteins that mediate insulin and cytokine action. Curr Top Microbiol Immunol 228:179-208
13. Kornmann M, Beger H G, Link K H (2003) Chemosensitivity testing and test-directed chemotherapy in human pancreatic cancer. Recent Results Cancer Res 161:180-195
14. Korc M (1998) Role of growth factors in pancreatic cancer. Surg Oncol Clin N Am 7:25-41
15. Kornmann M, Maruyama H, Bergmann U, Tangvoranuntakul P, Beger H G, White M F, Korc M (1998) Enhanced expression of the insulin receptor substrate-2 docking protein in human pancreatic cancer. Cancer Res 58:4250-4254
16. Bergmann U, Funatomi H, Kornmann M, Beger H G, Korc M (1996) Increased expression of insulin receptor substrate-1 in human pancreatic cancer. Biochem Biophys Res Commun 220:886-890
17. Basso D, Plebani M (2000) Cytokines and exocrine pancreatic cancer: is there a link? JOP 1:19-23
18. Hedin K E (2002) Chemokines: new, key players in the pathobiology of pancreatic cancer. Int J Gastrointest Cancer 31:23-29
19. Prokopchuk O, Liu Y, Henne-Bruns D, Kornmann M (2005) Interleukin-4 enhances proliferation of human pancreatic cancer cells: evidence for autocrine and paracrine actions. Br J Cancer 92:921-928
20. Kornmann M, Kleeff J, Debinski W, Korc M (1999) Pancreatic cancer cells express interleukin-13 and -4 receptors, and their growth is inhibited by Pseudomonas exotoxin coupled to interleukin-13 and -4. Anticancer Res 19:125-131
21. Liu Z, Neiss N, Zhou S, Henne-Bruns D, Korc, M, Bachem M, Kornmann M (2007) Identification of a fibroblast growth factor receptor 1 splice variant that inhibits pancreatic cancer cell growth. Cancer Res 67:2712-2719
22. Formentini A, Sander S, Denzer S, Straeter J, Henne-Bruns D, Kornmann M (2007) Thymidylate synthase expression in resectable and unresectable pancreatic cancer: role as predictive or prognostic marker? Int J Colorectal Dis 22:49-55
23. Pawankar R U, Okuda M, Hasegawa S, Suzuki K, Yssel H, Okubo K, Okumura K, Ra C (1995) Interleukin-13 expression in the nasal mucosa of perennial allergic rhinitis. Am J Respir Crit. Care Med 152:2059-2067
24. Ohshima K, Akaiwa M, Umeshita R, Suzumiya J, Izuhara K, Kikuchi M (2001) Interleukin-13 and interleukin-13 receptor in Hodgkin's disease: possible autocrine mechanism and involvement in fibrosis. Histopathology 38:368-375
25. Skinnider B F, Kapp U, Mak T W (2001) Interleukin 13: a growth factor in hodgkin lymphoma. Int Arch Allergy Immunol 126:267-276
26. Maini A, Hillman G, Haas G P, Wang C Y, Montecillo E, Hamzavi F, Pontes J E, Leland P, Pastan I, Debinski W, Puri R K (1997) Interleukin-13 receptors on human prostate carcinoma cell lines represent a novel target for a chimeric protein composed of IL-13 and a mutated form of Pseudomonas exotoxin. J Urol 158:948-953
27. Wright K, Kolios G, Westwick J, Ward S G (1999) Cytokine-induced apoptosis in epithelial HT-29 cells is independent of nitric oxide formation. Evidence for an interleukin-13-driven phosphatidylinositol 3-kinase-dependent survival mechanism. J Biol Chem 274:17193-17201
28. Arico S, Petiot A, Bauvy C, Dubbelhuis P F, Meijer A J, Codogno P, Ogier-Denis E (2001) The tumor suppressor PTEN positively regulates macroautophagy by inhibiting the phosphatidylinositol 3-kinase/protein kinase B pathway. J Biol Chem 276:35243-35246
29. Serve H, Oelmann E, Herweg A, Oberberg D, Serve S, Reufi B, Mucke C, Minty A, Thiel E, Berdel W E (1996) Inhibition of proliferation and clonal growth of human breast cancer cells by interleukin 13. Cancer Res 56:3583-3588
30. Blais Y, Gingras S, Haagensen D E, Labrie F, Simard J (1996) Interleukin-4 and interleukin-13 inhibit estrogen-induced breast cancer cell proliferation and stimulate GCDFP-15 expression in human breast cancer cells. Mol Cell Endocrinol 121:11-18
31. Obiri N I, Husain S R, Debinski W, Puri R K (1996) Interleukin 13 inhibits growth of human renal cell carcinoma cells independently of the p140 interleukin 4 receptor chain. Clin Cancer Res 2:1743-1749
32. Ihle J N (1996) Signaling by the cytokine receptor superfamily in normal and transformed hematopoietic cells. Adv Cancer Res 68:23-65
33. Rodriguez-Viciana P, Warne P H, Khwaja A, Marte B M, Pappin D, Das P, Waterfield M D, Ridley A, Downward J (1997) Role of phosphoinositide 3-OH kinase in cell transformation and control of the actin cytoskeleton by Ras. Cell 89:457-467
34. Curnock A P, Logan M K, Ward S G (2002) Chemokine signalling: pivoting around multiple phosphoinositide 3-kinases. Immunology 105:125-136
35. Wang L M, Michieli P, Lie W R, Liu F, Lee C C, Minty A, Sun X J, Levine A, White M F, Pierce J H (1995) The insulin receptor substrate-1-related 4PS substrate but not the interleukin-2R gamma chain is involved in interleukin-13-mediated signal transduction. Blood 86:4218-4227
36. Keegan A D, Nelms K, White M, Wang L M, Pierce J H, Paul W E (1994) An IL-4 receptor region containing an insulin receptor motif is important for IL-4-mediated IRS-1 phosphorylation and cell growth. Cell 76:811-820
37. Chiaramonte M G, Donaldson D D, Cheever A W, Wynn T A (1999) An IL-13 inhibitor blocks the development of hepatic fibrosis during a 1-helper type 2-dominated inflammatory response. J Clin Invest 104:777-785
38. Zhu Z, Horner R J, Wang Z, Chen Q, Geba G P, Wang J, Zhang Y, Elias J A (1999) Pulmonary expression of interleukin-13 causes inflammation, mucus hypersecretion, subepithelial fibrosis, physiologic abnormalities, and eotaxin production. J Clin Invest 103:779-788
39. Terabe M, Matsui S, Noben-Trauth N, Chen H, Watson C, Donaldson D D, Carbone D P, Paul W E, Berzofsky J A (2000) NKT cell-mediated repression of tumor immunosurveillance by IL-13 and the IL-4R-STAT6 pathway. Nat Immunol 1:515-520
40. Ostrand-Rosenberg S, Clements V K, Terabe M, Park J M, Berzofsky J A, Dissanayake S K (2002) Resistance to metastatic disease in STAT6-deficient mice requires hemopoietic and nonhemopoietic cells and is IFN-gamma dependent. J Immunol 169:5796-5804
41. Kawakami K, Kawakami M, Husain S R, Puri R K (2003) Potent antitumor activity of IL-13 cytotoxin in human pancreatic tumors engineered to express IL-13 receptor alpha2 chain in vivo. Gene Ther 10:1116-1128
42. Oshima Y, Puri R K (2001) A novel interleukin 13 (IL-13) antagonist that blocks the biological activity of human IL-13 in immune and nonimmune cells. FASEB J 15:1469-1471

1. Method for the diagnosis and/or prognosis of tumors, comprising the step of determining IL-13 expression and/or IL-4 receptor as diagnostic and/or prognostic marker in a patient.

2. The method of claim 1, wherein the tumors are solid tumors.

3. The method of claim 1, wherein the tumors are pancreas tumors.

4. The method of claim 1, wherein the tumors are colon tumors.

5. The method of claim 1, wherein high expression of IL-4 receptor and/or IL-13 is associated with a high incidence of lymph node involvement and/or a reduced overall survival.

6. Pharmaceutical composition comprising as an active agent an IL-4 receptor antagonist for the treatment of pancreas and/or colon tumors.

7. The pharmaceutical composition of claim 6, further comprising one or more pharmaceutically acceptable excipients.

8. The pharmaceutical composition of claim 6, further comprising one or more additional active substances.

9. The pharmaceutical composition of claim 8, wherein the additional active substance is an IL-13 antagonist.

10. The pharmaceutical composition of claim 6, wherein the IL-4 receptor antagonist is selected from the group consisting of IL-4 receptor antibodies, IL-4 muteins and/or other IL-4 receptor blocking agents.

11. Method for the treatment of pancreas and/or colon tumors comprising administering a therapeutically effective amount of an IL-13 antagonist and/or an IL-4 receptor antagonist to a patient.

12. Method for assessment of the longterm survival of an individual having a solid tumor comprising the steps of
(i) obtaining a sample of said solid tumor;
(ii) determining the level of expression of IL-13 and IL-4R in the tumor tissue; and
(iii) assessing the longterm survival of such individual based on the levels of expression of IL-13 and IL-4R in the tumor tissue,
wherein simultaneous expression of IL-13 and IL-4 at high levels in the tumor tissue is indicative of reduced longterm survival of the individual.
